(19) United States
(12) Patent Application Publication
(10) Pub. No.: US 2024/0424288 A1
(43) Pub. Date: Dec. 26, 2024

(54) INJECTABLE SELF-EXPANDING ELECTRODES WITH DISCONTINUOUS ELEMENTS

(71) Applicant: Micro-Leads, Inc., Somerville, MA (US)

(72) Inventors: Keith Angelino, Cambridge, MA (US); Mark Baldiswieler, Brookline, MA (US); Bryan L. McLaughlin, Cambridge, MA (US)

(21) Appl. No.: 18/647,887

(22) Filed: Apr. 26, 2024

Related U.S. Application Data
(60) Provisional application No. 63/523,216, filed on Jun. 26, 2023.

(51) Int. Cl. A61N 1/05; A61N 1/36; A61N 1/375; A61N 1/378

(52) U.S. Cl. CPC A61N 1/05; A61N 1/3605; A61N 1/3754; A61N 1/3787

(57) ABSTRACT

An electrophysiology lead system has a lead including a longitudinal body, a substrate, and a therapy electrode. The lead has an extended configuration and a compressed configuration. The substrate is normally biased toward the extended configuration. The electrode delivers an electrical signal, the electrode has a nonuniform thickness or a discontinuity. The electrode compresses when the lead is in the compressed configuration. The electrode is configured so that the substrate urges the electrode toward the extended configuration. The electrode has a thickness between 2 microns and 200 microns.

### PRIORITY

This patent application claims priority from provisional U.S. patent application No. 63/523,216, filed Jun. 26, 2023, entitled, “INJECTABLE SELF-EXPANDING ELECTRODES WITH DISCONTINUOUS ELEMENTS,” and naming Keith Angelino et al. as inventors, the disclosure of which is incorporated herein, in its entirety, by reference.

### FIELD

Illustrative embodiments of the invention generally relate to electrophysiology leads and, more particularly, various embodiments of the invention relate to self-expanding electrophysiology leads.

### BACKGROUND

Electrophysiological leads may be inserted into an anatomical body for the purposes of diagnosis or treatment. For example, a lead may be used to stimulate or monitor neural tissue, among other things. By implanting the lead adjacent to the neural tissue, the lead can transceive electrical signals with the neural tissue. Existing implantable leads that can be delivered through a sheath or needle treat a small volume of tissue using a cylindrical stimulation field. Furthermore, implantable leads may also stimulate/monitor electrical signals using a unidirectional field transceiving signals not only with a target neural tissue, but also the surrounding tissue. Implantable leads may be separated from a stimulator by a connector or the lead may be fully-integrated with a stimulator to deliver therapy.

### SUMMARY OF VARIOUS EMBODIMENTS

In accordance with one embodiment of the invention, an electrophysiology lead system has a lead including a longitudinal body, a substrate, and a therapy electrode. The lead has an extended configuration and a compressed configuration. The substrate is normally biased toward the extended configuration. The therapy electrode delivers an electrical signal, the electrode has a nonuniform thickness or a discontinuity. The electrode compresses when the lead is in the compressed configuration. The electrode is configured so that the substrate urges the electrode toward the extended configuration. The electrode has a thickness between 2 microns and 200 microns.

The electrode nonuniform thickness may have an indentation extending partly through the thickness of the electrode. The discontinuity may have a void in the electrode extending completely through the thickness of the electrode forming a distributed electrode.

The normal bias of the substrate may be a function of elasticity, shape-memory behavior, thermal change, phase change, polymeric change, or volumetric change.

In some embodiments, the substrate includes a guide to direct the lead into the compressed configuration in response to the lead being directed into a lumen.

The lead may include a plurality of therapy electrodes including the first electrode. The plurality of therapy electrodes may be oriented in the same direction when the lead is in the extended configuration.

In some embodiments, the substrate includes an anchor configured to mechanically couple the lead to a biological tissue.

In some embodiments, the electrophysiology lead system includes a radiopaque marker coupled to the substrate.

In accordance with another embodiment of the invention, a method for electrically coupling a lead and neural tissue directs the lead into a compressed configuration including compressing the electrode of the lead. The electrode has a nonuniform thickness or a discontinuity. The electrode delivers an electrical signal and includes a thickness between about 2 microns and 200 microns. The method then transitions the lead from the compressed configuration to an extended configuration, using a substrate of the lead including extending the electrode. The substrate is normally biased toward the extended configuration.

The nonuniform thickness may have an indentation extending partly through the thickness of the electrode. The discontinuity may comprise a void in the electrode extending completely through the thickness of the electrode forming a distributed electrode.

The normal bias of the substrate may be a function of elasticity, shape-memory behavior, thermal change, phase change, polymeric change, or volumetric change.

Directing the lead into the compressed configuration may include directing, using a guide, the lead into the compressed configuration in response to directing the lead into a lumen.

The lead may have a plurality of electrodes including the first electrode. Transitioning the lead may include at least a portion of the plurality of electrodes orienting in the same direction.

In some embodiments, the method couples the lead to a biological tissue using an anchor of the substrate. In another embodiment, the method couples the lead to a biological tissue using an anchor of the lead body.

In some embodiments, the substrate may have a radiopaque marker.

In accordance with another embodiment of the invention, an electrophysiology lead has a longitudinal body, a substrate, and an electrode. The substrate is biased to move the electrode from a compressed configuration to an extended configuration. The electrode has a plurality of bending zones configured to be flexed in the compressed configuration and relaxed as the substrate moves toward the extended configuration. The electrode includes a thickness between about 2 microns and 200 microns.

The plurality of bending zones includes an indentation extending partly through the thickness of the electrode, or a void in the electrode extending completely through the thickness of the electrode.

The normal bias of the substrate may be a function of elasticity, shape-memory behavior, thermal change, phase change, polymeric change, or volumetric change.

In some embodiments, the substrate includes a guide to direct the lead into the compressed configuration in response to directing the lead into a lumen.

In some embodiments, the lead includes a plurality of electrodes including the first electrode, each of the plurality of electrodes being oriented in a same direction when the lead is in the extended configuration.

In some embodiments, the substrate has an anchor to couple the lead to a biological tissue.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIGS. 1A-1B are cross-sectional views schematically showing an electrophysiology lead in accordance with various embodiments.

FIG. 2 is a top view schematically showing an electrophysiology lead system in accordance with various embodiments.

FIGS. 3A-3B are cross-sectional views schematically showing electrophysiology leads in accordance with various embodiments.

FIGS. 4A-4B are cross-sectional views schematically showing substrates of the electrophysiology leads in accordance with various embodiments.

FIG. 5A-E schematically show substrate components in accordance with various embodiments.

FIGS. 6A-E schematically show substrate mechanical anchors in accordance with various embodiments.

FIG. 7 schematically shows substrate guides in accordance with various embodiments.

FIGS. 8A-8D schematically show electrode array geometric configurations in accordance with various embodiments.

FIGS. 9A-9B schematically show nonuniform thickness indentions of an electrode in accordance with various embodiments.

FIGS. 10A-11B schematically show nonuniform thickness and discontinuities of an electrode in accordance with various embodiments.

FIG. 12 shows alignment of radiopaque markers in accordance with various embodiments.

FIG. 13 schematically shows a lumen with markers in accordance with various embodiments.

FIGS. 14A-C schematically show leads and substrates with markers in accordance with various embodiments.

FIG. 15 is a flowchart showing a process for coupling a lead and a neural tissue.

FIGS. 16A-D show a sequence for decompressing an implanted lead in accordance with various embodiments.

FIG. 17 shows a lead system with a lead connected to a controller, a lead integrated with a controller, and a wireless electrophysiology lead system in accordance with various embodiments.

### DESCRIPTION OF ILLUSTRATIVE EMBODIMENTS

In illustrative embodiments, an electrophysiology lead is compressed into a lumen and implanted into an anatomical body. While positioned and oriented proximate to a target tissue, the lumen is removed, allowing the lead to expand, forming a surface with electrodes directed towards the target tissue and configured to generate a directed electric field toward the target tissue. Details of illustrative embodiments are discussed below.

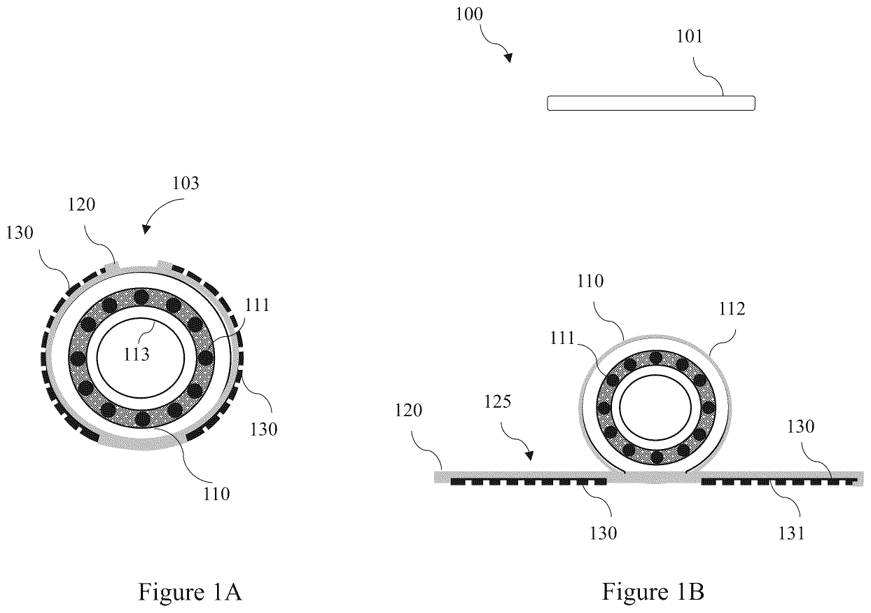

FIGS. 1A and 1B are cross-sections of an electrophysiology lead 103 configured to receive electrical signals from or transmit electrical signals to a target anatomical tissue. The lead 103 is one component of an electrophysiology lead system 100 configured to implant and operate the lead 103. The lead system 100 may be used in a variety of applications, including stimulation or recording from different parts of an anatomical body including the spinal cord, brain, sacral nerve, peripheral nerve, vagus nerve, carotid sinus nerve, or upper airway patency nerve, among other things. The lead system 100 may also be used for cardiac resynchronization or anatomical monitoring, among other things.

It should be appreciated that the length of a component refers to the largest dimension of the component. The longitudinal direction of the component is parallel with the length of the component and orthogonal to the width of the component. The width of a component of the lead system 100 refers to the second largest dimension of the component. The latitudinal direction of the component is parallel to the width of the component and orthogonal to the length of the component. The thickness of a component of the lead system 100 refers to the smallest dimension of the component, orthogonal to both the length and the width.

The lead 103 includes a longitudinal body 110 configured to conduct an electrical signal transmitted between the lead 103 and the target neural tissue. The longitudinal body includes a conductor 111 configured to conduct the electrical signal from the source to the electrode 130. In the illustrated embodiment, the conductors 111 are arranged around a central insulator 113. In other embodiments, the conductors may be arranged within a cross-section of a different shape or relative position to one another (e.g. braided, helical, adjacent, etc.). The conductors 111 may be housed within an insulative layer 112 which may be configured to isolate the conductors 111 from the tissue. The longitudinal body may also include a second inner insulative layer 113 configured to isolate the conductor 111 from objects placed within the central lumen of the longitudinal body 110. In some embodiments, the conductor 111 does not extend the length of the longitudinal body 110, in which case the conductor 111 is coupled to wires, or other conductive structures, located in the central opening of the longitudinal body 110.

The electrophysiology lead 103 includes wings 125 having electrodes 130 configured to transition between a compressed configuration and an extended configuration. FIG. 1A shows the lead 103 arranged in a compressed configuration, also known as a delivery configuration. In the compressed configuration, the wings 125 are compressed around the longitudinal body 110 in order to reduce a cross-sectional dimension of the lead 103. The wings 125 may be compressed by curling, folding, or another action which reduces a dimension of the wings 125. The lead 103 may be in the compressed configuration while the lead 103 is being delivered to the implant anatomy. FIG. 1B illustrates the lead 103 in the extended configuration. In the extended configuration, also known as the operational configuration, the wings 125 increase the surface area dimensions interfacing with a target neural tissue. The wings 125 may extend by uncurling, unfolding, or another action which increases a dimension of the wings 125. The lead 103 may be transitioned from the compressed configuration to the extended configuration after the lead 103 is inserted into the body and positioned/oriented proximate to the target neural tissue.

The lead 103 includes a substrate 120 extending from the longitudinal body 110, the substrate 120 forming the wings 125. The substrate 120 transitions between the compressed configuration and the extended configuration. The substrate is normally biased towards the extended configuration. The normal bias of the substrate 120 may be a function of elasticity, shape-memory behavior, thermal change, phase change, polymeric change, or volumetric change, among other things. In the extended configuration, the substrate 120 has an increased surface area dimension to interface with a target tissue. In some embodiments, the cross-sectional area of the substrate 120 may vary along the length of the substrate 120.

In the extended configuration, the substrate 120 has a width substantially greater than the diameter of the longitudinal body 110. For example, the substrate may have a width at least 30% or at least 50% greater than the diameter of the longitudinal body 110. In some embodiments, the width of the substrate 120 is at least three times greater than the width of the longitudinal body 110.

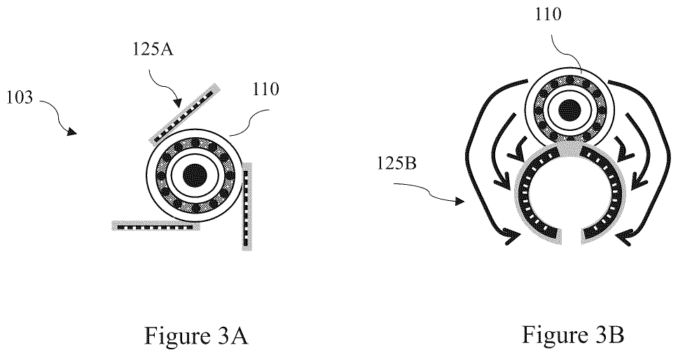

The lead 103 includes a therapy electrode 130, herein also referred to as an electrode, electrically coupled to the conductor 111, and configured to deliver an electrical signal between the conductor 111 and a target tissue. The substrate 120, when in the extended configuration, may form a surface upon which a plurality of electrodes 130 may be positioned and oriented such that the plurality of electrodes 130 are oriented in the same direction. By orienting electrodes 130 in the same direction, such as towards a target neural tissue, the electrodes 130 generate a preferred stimulation field direction. A preferred stimulation field direction may be substantially planar (FIG. 1B) or substantially curved (FIG. 3B).

The electrode 130 is distributed so that it is comprised of conductive sections directly contacting a target neural tissue, with the sections being coupled together by bending zones 131 which aid the electrode 130 in transitioning between the compressed configuration and the extended configuration. The bending zones 131 are configured to be flexed in the compressed configuration and relaxed in extended configuration. The bending zones 131 enable the compression of the electrode 130 to be focused and distributed among a portion of the electrode 130. The bending zones 131 may correspond to a portion of the electrode having a reduced thickness, as shown in FIG. 1B. The reduced thickness may be an indentation or a void. The benefits of the bending zones 131 include: (1) reducing damage or deformation of the electrode 130 when compressed during implantation, and (2) the electrode may still have sufficient thickness to support high break-strength welded connections to wires for long-term implanted reliability.

As shown in FIG. 1B, the electrodes 130 may be oriented in the same direction while the lead 103 is in the extended configuration. The therapy orientation allows for a directed stimulation field into the tissue interfacing with the electrode 130. This is particularly important for focusing stimulation or recording voltage potentials of a specific area. The directionality of the electrodes 130 also provides more efficient use of stimulation, a lower dose of power to achieve the same therapeutic efficacy, and a reduced stimulation of off-target directions.

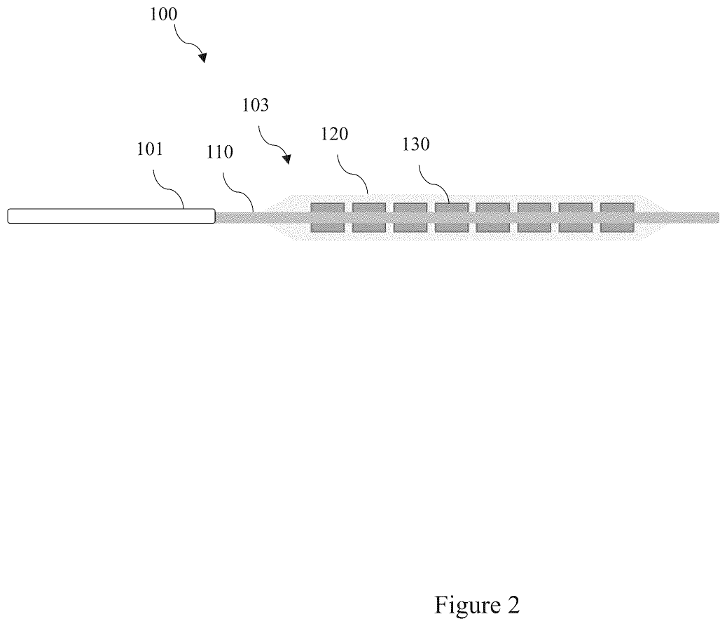

FIG. 2 is a top view of an electrophysiology lead system 100 including the lead 103. As illustrated, the lead 103 is partially inserted into a lumen 101 configured to house the lead 103. Among other things, the lumen 101 may include an introducer sheath, an introducer needle, a cannula, a catheter, guidewire, or steering stylet. The lumen 101 may be straight, bent, or curved, among other things. The lumen 101 includes an opening into which the lead 103 may be partially or fully inserted. The opening in the lumen 101 has a cross-section perpendicular to the length of the opening. The shape of the cross-section of the lumen 101 may be circular, elliptical, square, rectangular, triangular, or hexagonal, polygonal, among other things. In some embodiments, the diameter of the lumen 101 is less than 3.0 mm.

As shown in FIG. 2, the lead 103 may include multiple therapy electrodes 130. In some embodiments, the electrodes 130 are electrically coupled by way of the longitudinal body 110 such that the same voltage/current signals over all electrodes 130 simultaneously. In some embodiments, the electrodes 130 are configured to transmit different electrical signals simultaneously. In some embodiments, a portion of the electrodes 130 may transmit an electrical signal while the remaining electrodes 130 do not. Stimulation electrodes may also comprise anode, cathodes, and ground returns.

FIG. 3A is a cross-sectional view showing a multi-wing configuration 125A in an extended configuration. The multi-wing configuration includes more than two wings, each of which may extend from the cross-section of the longitudinal body 110. Each wing may be configured to be compressed around the longitudinal body 110 in the compressed configuration. As the wings transition from the compressed configuration to the extended configuration, each wing becomes tangential to the cross-sectional point of coupling. Therapy electrodes 130 in the multi-wing configuration form a directional field that points in multiple directions.

FIG. 3B is a cross-sectional view showing a wing configuration 125B having an encircling extended configuration. The wing configuration 125B has wings configured to be compressed around the longitudinal body when in the compressed configuration. As the wings transition to the extended configuration, the wings 125B encircle a point outside of the longitudinal body 110, surrounding a target tissue (e.g., a peripheral nerve). Therapy electrodes in the encircling configuration form a directional field that points in an encircled direction.

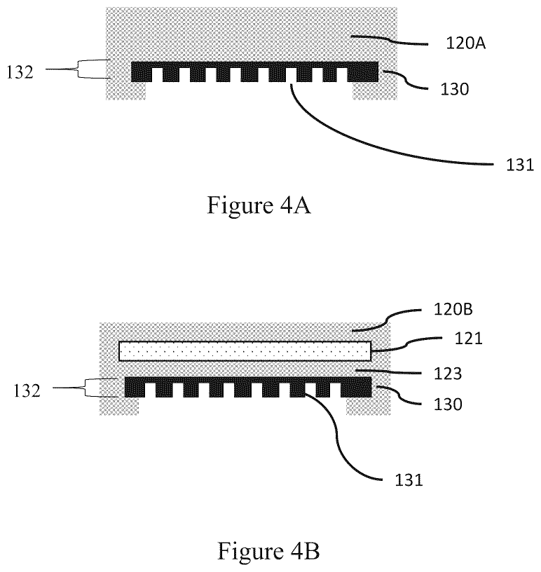

FIG. 4A is a cross-sectional view of the substrate 120A and the electrode 130 of the lead 103. The substrate 120A is homogenous, comprised of a single material without additional layers where the substrate material comprises an urging material. In some embodiments, the substrate 120A is be electrically insulative.

The electrode 130 has a thickness 132. In some embodiments, the thickness 132 is between 2 microns and 200 microns. The electrodes 130 may be formed of noble metals or noble metal coatings for implantable stimulation and recording (e.g., platinum, platinum-iridium, rhodium, iridium-oxide, or coated nickel-titanium alloy, nitinol, among other things).

The electrode 130 includes bending zones 131 configured to allow the electrode to be compressed without creasing the electrode. In the illustrated embodiment, the bending zones are formed by a nonuniform thickness of the electrode 130.

FIG. 4B is a cross-sectional view of the substrate 120B and the electrode 130. Unlike the homogenous urging substrate 120A of FIG. 4A, the substrate 120B includes an embedded urging layer 121 configured to bias the substrate 120B and the electrode 130 toward the extended configuration and away from the compressed configuration. The embedded urging layer 121 may be comprised of elastic, super-elastic, or shape-memory material. In the illustrated embodiment, the urging layer 121 is a solid layer. In other embodiments, the urging substrate may be comprised of a patterned layer, frame, or isolated layer features, among other things to tune the urging properties. In another embodiment, the electrode may be comprised of nitinol and feature an electrical coating of an inert noble metal (e.g., platinum-iridium)

The urging layer 121 may be surrounded by an electrically insulating material 123. The material 123 may be comprised of silicone, polyimide, PTFE, PET, LCP, PEEK, PU, TPU, PEBA, or PEBAX, among other things. The urging layer 121 may be a different material than the electrically insulating material 123, or the same material. For example, the electrically insulating material 123 may be a flexible polymer such as polyurethane (e.g., polyimide, PTFE, PET, LCP, PEEK, PEBA) with a metallic urging element (e.g., nickel-titanium, Nitinol).

In some embodiments, as illustrated embodiment in FIGS. 4A and B, the electrode 130 is embedded in the substrate 120A, 120B to encapsulate the side of the electrode 130 and focus stimulation or recorded signals through the electrode 130 in a uniform direction.

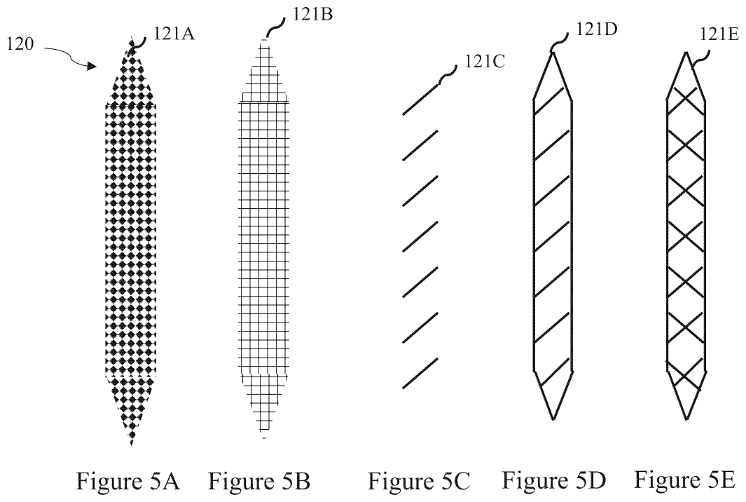

FIGS. 5A-E show some of the structural embodiments of the urging layer 121. As illustrated in FIG. 5A, an urging layer 121A may be homogeneous (FIG. 4B, 121) or have a perforated structure (FIG. 5A). As illustrated in FIG. 5B, an urging layer 121B may have a mesh or woven structure. As illustrated in FIG. 5C, an urging layer 121C may be comprised of discrete elements. As illustrated in FIGS. 5D and 5E, urging layers 121D or 121E may be comprised of a frame, spine, or other support features. More than one urging layer may be used.

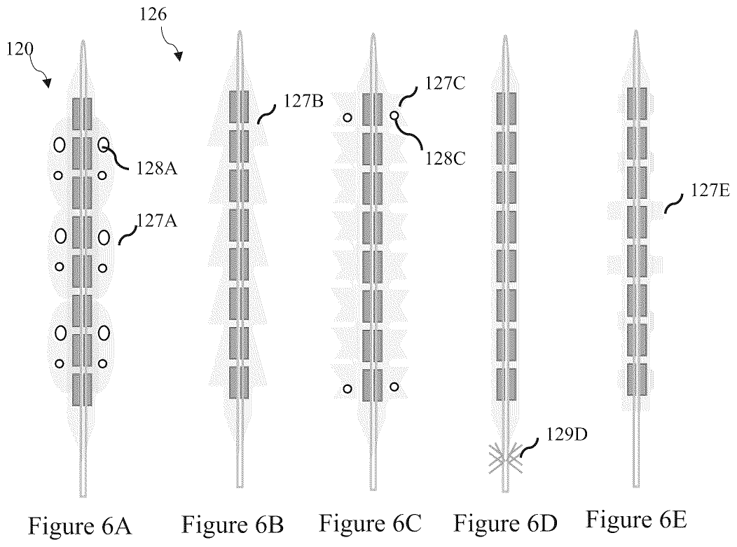

FIGS. 6A-6E shows substrates 120 having multiple anchor configurations 126 to mechanically couple the lead 103 to a target tissue. Mechanical coupling may prevent the electrode from migrating and to maintain a stable coupled with a target tissue (e.g. during movement). It should be appreciated that the lead 103, when in the expanded configuration, has a greater resistance to migration relative to other types of leads, such as a lead with a cylindrical cross-section. The anchor configurations 126 are additional features to further constrain movement of the lead 103 relative to the target tissue, also known as migration, in one or more longitudinal direction or more or more latitudinal directions.

The anchor configuration 126 may include an edge feature 127 to resist migration of the lead 103 as the tissue moves, or when a force is exerted on the lead system after an implantation procedure. Among other things, the edge feature 127 may include a curved edge, such as edge feature 127A in FIG. 6A; a sawtooth edge (i.e., diagonal features facing one direction), such as edge feature 127B in FIG. 6B; a bidirectional sawtooth edge (i.e., diagonal features facing two directions), such as edge feature 127C in FIG. 6C; or rectangular protrusion edge features of varying lengths, such as edge feature 127E in FIG. 6E. The edge features 127 may resist migration in one direction or multiple directions.

The edge feature 127 of the substrate 120 may be selected based on the specific application of the lead 103. For example, the edge feature 127B may be selected to allow the lead 103 to be inserted without resistance, but resist the extraction of the lead 103 once inserted. Furthermore, the edge features 127C and 127E may be selected to resist both longitudinal and latitudinal migration. As another example, the edge feature 127A may be selected to resist migration while reducing nerve or tissue irritation compared to other edge features. The substrate 120 may include a number or total length of edge features 127 based on a desired magnitude of migration resistance, such as a desired pull resistance. The edge feature may also be designed to enable the lead to be positioned within a lumen for implanting or removing the lead.

The anchor configuration 126 may include a hole feature 128 configured to provide a point at which the lead 103 may be coupled to tissue by way of a suture connection. The hole feature 128 may also be configured to allow for tissue growth through the substrate 120, allowing the tissue growth to affix the lead 103 to the biological tissue. As illustrated in FIG. 6A, the substrate 120 may include hole features 128A of varying shapes or diameters. As illustrated in FIG. 6C, the substrate 120 may include hole features 128C of uniform shape or width. The substrate 120 may include a number of hole features 128 based on a desired magnitude of migration resistance, such as a desired pull resistance.

The anchor configuration 126 may include tines configured to resist migration of the lead 103. The tines may be located on the longitudinal body 110. As illustrated in FIG. 6D, tines 129D may be located near the proximal end of the substrate 120. The tines may be located at other points along the longitudinal body 110, such as near the distal end of the substrate, among other things.

It should be appreciated the lead 103 may have anchor configurations 126 using one or more of the features illustrated herein. For example, the anchor configurations may combine at least two of: edge features, hole features, or tines.

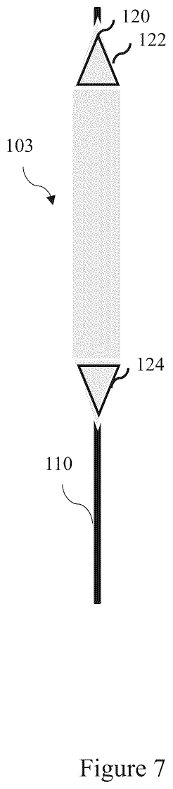

FIG. 7 shows the substrate 120 having geometric guides 122, 124 configured to direct the lead 103 into the compressed configuration in response to the lead 103 being directed into the lumen 101 or anatomical tissue. In the illustrated embodiment, the substrate 120 includes a proximal end guide 124 and a distal end guide 122. As the lead 103 is pushed into the lumen 101 (i.e., inserted distal end first), the distal end guide 122 directs the substrate 120 to compress to conform to the shape of the inside of the lumen 101. As the lead 103 is pulled into the lumen 101 (i.e., inserted proximal end first), the proximal end guide 124 directs the substrate to compress to conform to the shape of the inside of the lumen 101. The guides may also help return the substrate to the extended configuration when exiting the lumen 101 or biological tissue.

The guides 122, 124 have a length and a width geometry which tapers approaching a tip of the guide 122, 124. In the illustrated embodiment, the guides 122, 124 include a solid material. Among other things, the guide 122, 124 may have an urging frame defining edges of the guide, or a spine parallel with the longitudinal body 110. The selection of an urging or non-urging material solid form, frame, or spine may be determined by a desired force magnitude to insert or retract the substrate 120 from the lumen 101, among other things. The length or width of the guides 122, 124 may also be configured to a desired insertion or retraction force. For example, as the length of the guide increases, the required insertion or retraction force may decrease.

The guides 122, 124 may be comprised of an urging material or layer 121. The guides may be a solid material or may be comprised of a wire, mesh, foil, honeycomb, or frame. The guides 122, 124 may be comprised of a layer of shape-memory urging material (e.g., nickel-titanium, Nitinol, or polymeric materials (e.g., polyimide, PTFE, PET, LCP, PEEK, PEBA), among other things. The guides 122, 124 may be stamped, die-formed, laser-cut, etched, or formed using a variety of processes.

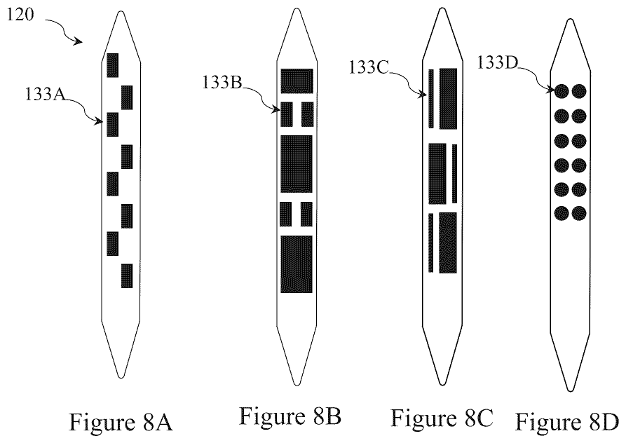

FIGS. 8A-8E show electrode arrangements according to various embodiments. As shown in FIG. 8A, the electrodes may be arranged in a staggered configuration in the substrate 120. As shown in FIGS. 8B and 8C, electrode configurations 133B, 133C may include electrodes of varying lengths or widths. As shown in FIG. 8D, the electrode configuration 133D may include non-rectangular shaped electrodes 130, such as electrodes in the shape of a circle, among other things.

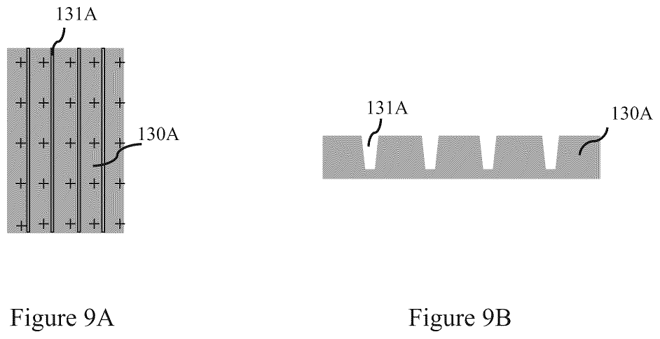

FIG. 9A is a top view of the electrode 130A having a nonuniform thickness including a plurality of indentations 131A extending partially through the thickness of the electrode 130A. FIG. 9B is a side view of the electrode 130A. The depth of the indentations 131A is configured to allow the lead 103 to compress into the lumen 101. In the illustrated embodiment, the indentations 131A are beveled to allow the electrode 130A to compress into the lumen 101. In some embodiments, the indentations 131A may be chamfered or straight edge, among other things.

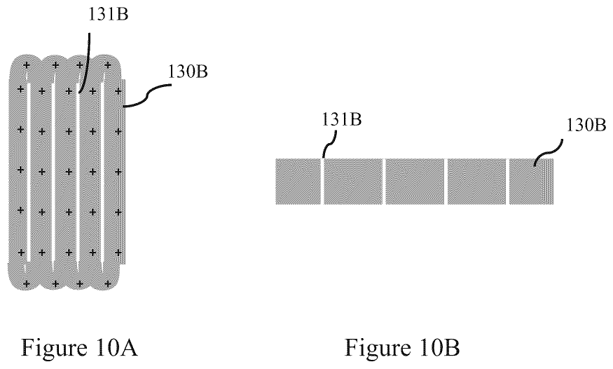

FIG. 10A is a top view of an electrode 130B having discontinuities including a plurality of voids extending through the electrode 131B arranged in a parallel configuration through the center of the electrode 130B. FIG. 10B is a cross-sectional view of the electrode 130B. The voids 131B are configured to allow the lead 103 to compress into the lumen 101.

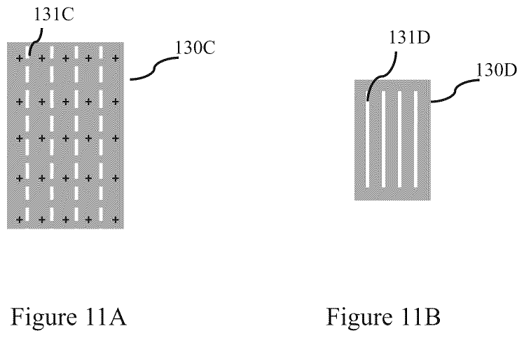

FIGS. 11A and 11B show additional embodiments of the electrode 130, each of the embodiments including discontinuity voids configured to allow the electrode 130 to be compressed into the lumen 101. FIG. 11A include voids 131C in the form of series of perforations arranged in parallel lines. FIG. 11B includes voids 131D in the form of parallel line-like voids through a center of the electrode 130D. In other embodiments, the voids 131D may form curved or wavey line-like voids.

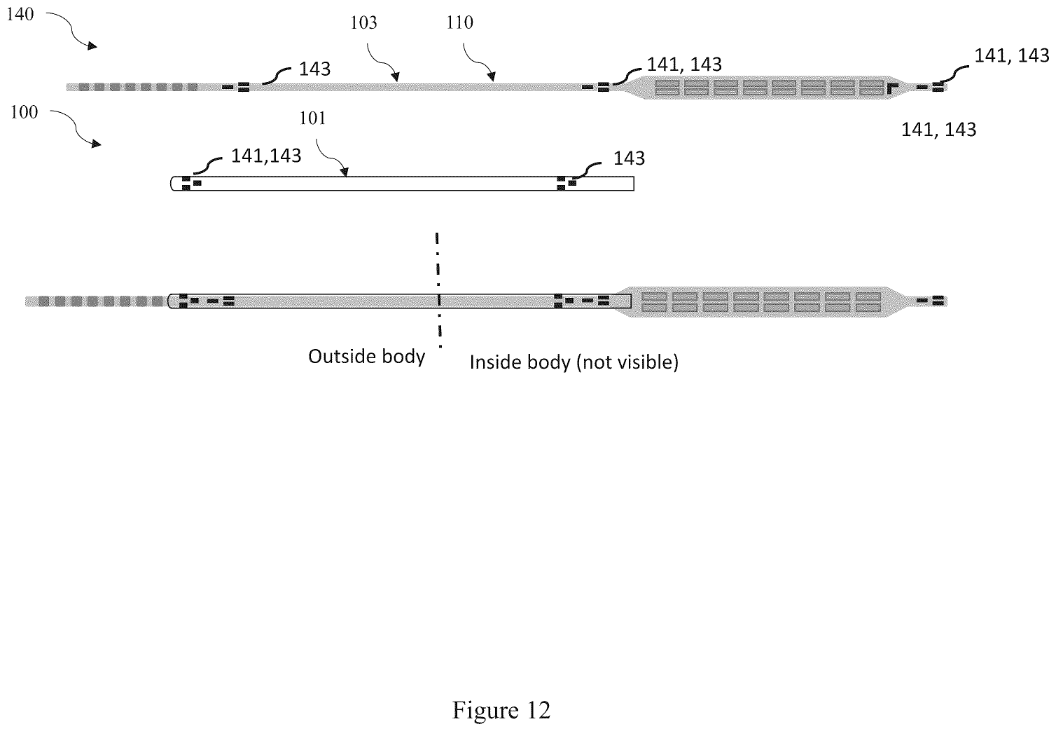

FIG. 12 shows an alignment of the lead 103 with the lumen 101 in accordance with various embodiments. As illustrated in FIG. 12, the lead system 100 may include a plurality of markers 140 (which include visible markers 143 and radiopaque markers 141) to assist the user with implanting the lead 103 in the correct orientation.

The markers 140 may be located on the substrate 120, the longitudinal body 110, or the lumen 101, a needle, among other things. In some embodiments, the markers 140 may include a marker visible to the human eye. In some embodiments, the marker may be radiopaque, visible using radiation-based imagery. The markers 140 may include other types of markers, such as echogenic markers, among other things. As in the illustrated embodiment, the plurality of markers 140 may include a combination thereof.

The illustrated lead system 100 includes visible markers 143 located on the proximal side of the longitudinal body 110 corresponding to visible markers located on the lumen 101. While the lead 103 is inserted into the lumen 101, the user may axially align the markers 143 on the lead 103 with the markers 143 on the lumen 101 to establish the orientation or depth of the lead 103.

The illustrated lead system 100 also includes radiopaque markers 141 located on both the proximal and distal sides of the longitudinal body 110. While inserted into an anatomical body, certain types of markers 140, such as radiopaque or echogenic markers, may be used to visualize the position and orientation of the lead 103. These markers may also be used to confirm the lead 103 has transitioned from the compressed configuration to the expanded configuration. The substrate may also include radiopaque markers or the electrodes themselves may be radiopaque to be able to identify the position of the electrode system during the implantation procedure.

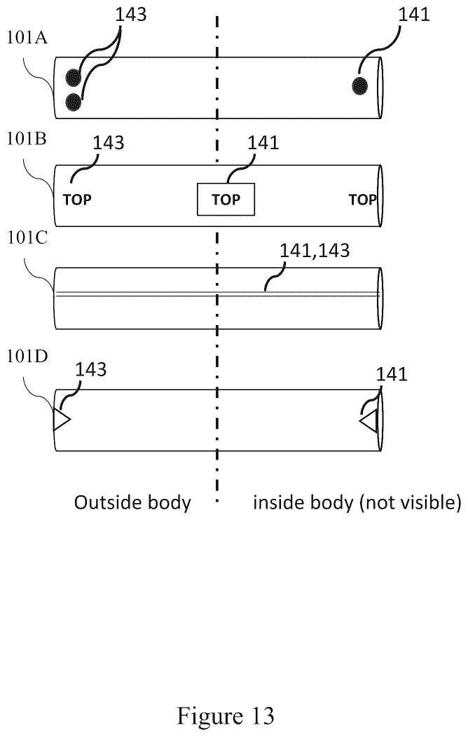

FIG. 13 schematically shows various embodiments for markers on the lumen 101. The lumen 101A illustrates how a visible lumen orientation marker 143 outside the body is paired with an electrode marker 141 inside the body. With paired markers inside and outside the body, the lead 103 can be oriented with respect to the visible marker 143. Once inside the body, the visual inspection of the marker 143 outside the body can be directly viewed, with or without the need for additional equipment, to identify the position/orientation of the lead 103 inside the body. As shown in the illustrated embodiments, dots/holes (lumen 101A), text (lumen 101B), internal wire coloration (lumen 101C), or notches (FIG. 46D), among other things, may also be used to identify the position/orientation of the lead 103 inside the body by observing markers outside the body.

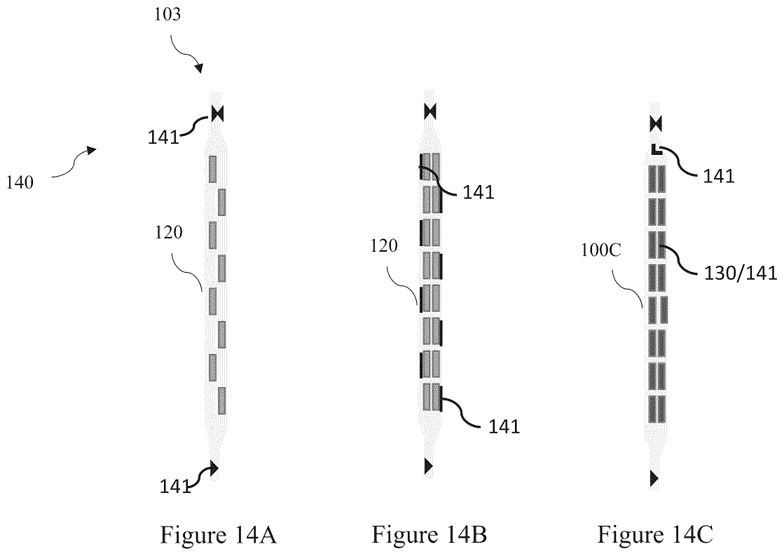

FIGS. 14A-14C schematically show markers 140 in different locations on the lead 103. The markers 140 are configured to assist a user with positioning and orienting the lead 103 in an anatomical body. FIG. 14A shows radiopaque markers on the proximal and distal ends of the longitudinal body 110. FIG. 14B shows radiopaque markers 141 located on, or embedded within, the substrate 120. Because the radiopaque markers are positioned towards the edges of the substrate, radiation imaging may be used to confirm the substrate successfully transitioned from the compressed configuration to the extended configuration. FIG. 14C shows an embodiment where the electrodes 130 are themselves radiopaque markers 141, being comprised of a material to enable visualization under fluoroscopy or ultrasound, among other things.

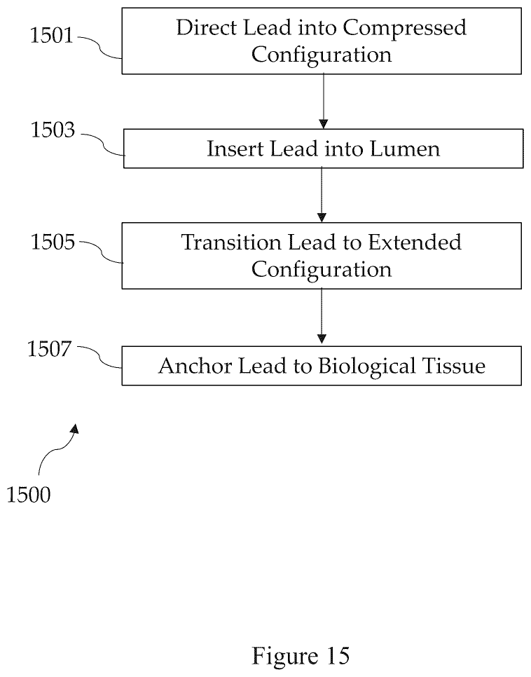

FIG. 15 shows an exemplary process 1500 for coupling the lead 103 and a target neural tissue. The Process 1500 may be implemented in whole or in part using one or more of the electrophysiology lead systems 100 disclosed herein. It shall be further appreciated that a number of variations and modifications to the Process 1500 are contemplated including, for example, the omission of one or more aspects of the Process 1500, the addition of further conditionals and operations, or the reorganization or separation of operations and conditionals into separate processes.

The Process 1500 begins directing the lead 103 into the compressed configuration in operation 1501. When in the compressed configuration, the substrate 120 and the electrodes 130 are compressed. Among other things, the substrate 120 and electrodes 130 may be curled around the circumference of the longitudinal body 110, or may be compressed to reduce the width of the lead 103. The lumen 101 may be configured to resist the normal biasing of the lead 103 toward the extended configuration. As the lead 103 is compressed, each of the electrodes 130 change shape along with the substrate. The electrode 130 may be reshaped by flexing at the bending zones 131, which are sections of the electrode 130 configured to be flexed to reduce the size of the lead 103 while being implanted.

Directing the lead 103 into the compressed configuration may include inserting the lead 103 into the lumen 101 where the width or diameter of the lumen 101 is less than the extended width of the lead 103. In some embodiments, one of the guides 122, 124, when compressed by the lumen 101, initiates the compression of the lead 103.

After the lead 103 is positioned in the lumen 101, the Process 1500 inserts the lumen 101 and lead 103 into an anatomical body in operation 1503. Inserting the lumen 101 and lead 103 may include steering the lumen 101 and lead 103 to a position and orientation relative to the target tissue.

In some embodiments, the user positions or orients the lead 103 relative to the target tissue using one or more markers 140. Among other things, using fluoroscopy, radiography, ultrasound or other imaging techniques, the lead 103 may be advanced towards the desired therapy location. For example, a stylet or guidewire may be used to steer the distal end of the lead as it is being advanced. Alternatively, a dilator and introducer catheter may be used to advance the lead 103 to the desired target.

After positioning and orienting the lead 103 and lumen 101, the Process 1500 transitions the lead 103 from the compressed configuration to the extended configuration. In some embodiments, transitioning the lead 103 to the extended configuration includes retracting the lumen 101, thus removing the lumen 101 from the lead 103. Without the lumen 101, the normal bias of the wings 125 move the wings towards the extended configuration, and the lead 103 begins to transition from the compressed configuration to the extended configuration. In another embodiment, the lumen 101 may be a tear-away or peelable introducer facilitating the removal of the lumen 101 from the lead after being implanted by peeling or splitting the introducer into removable pieces after implantation. It should be noted that after the lead transitions to the extended configuration 1505, the lead may be re-inserted into the lumen for optimal coupling to biological tissue followed by transitioning again to the extended configuration. In some embodiments, when fully retracted, the substrate 120 forms a surface, and the electrodes 130 are oriented in the same direction. The surface may be a larger treatment area (e.g., 3-4× wider than the cover 101 diameter). The electrodes 130 may then provide directional therapy, electrically coupling to the tissue.

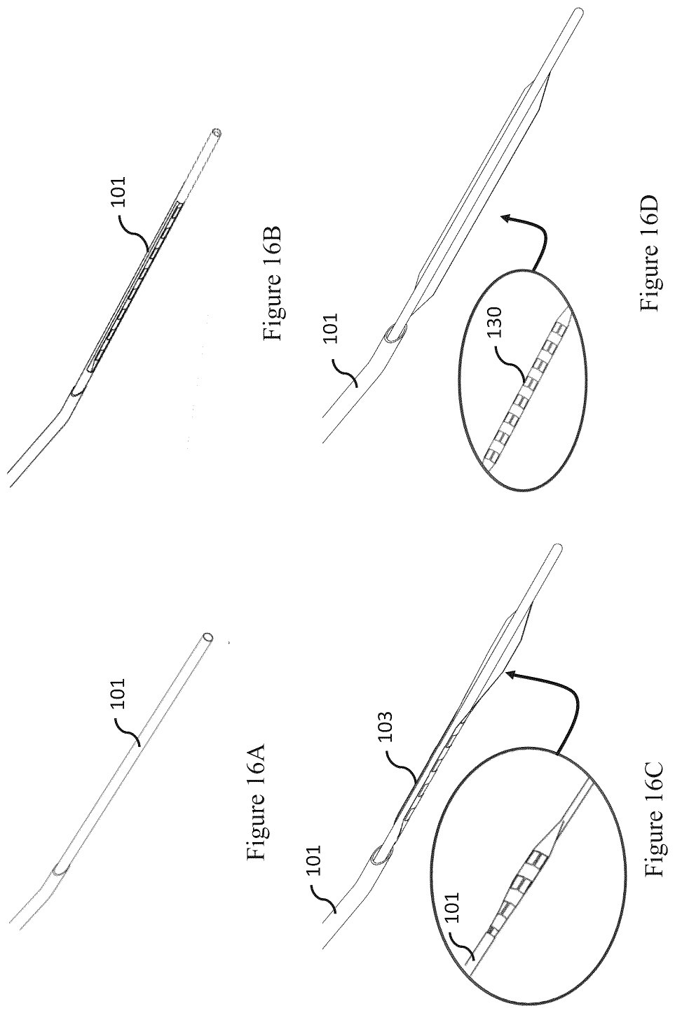

In the extended configuration, the lead 103 may begin to anchor to the surrounding tissue in operation 1507. The lead 103 may include one or more anchors 126 to resistant latitudinal, rotational, or longitudinal movement. In some embodiments, anchoring the lead 103 may include edge features of the substrate 120 interfacing with surrounding tissue, or tissue growing through holes in the substrate, among other things. FIGS. 16A-16D shows a sequence of steps for implanting the lead 103 proximate to a target tissue in accordance with various embodiments. In the illustrated embodiment, the lead 103 has been guided into the lumen 101 comprising a sheath inserted into a bent needle. The sheath may remain inserted inside the needle until the needle has been inserted into the anatomical body and positioned near the target tissue. After the needle is positioned, the sheath is urged out of the needle and positioned proximate to the target tissue, as illustrated in FIG. 16A.

FIG. 16B shows the position of the lead 103 relative to the needle and sheath, the sheath shown here as transparent, after the sheath is urged out of the needle. While inside the sheath, the lead 103 is in the compressed configuration. After the lead 103 is positioned proximate to the target tissue, the sheath is retracted while the lead 103 remains in position. As the sheath retracts, the lead 103, which is normally biased towards the extended configuration, begins to transition toward the extended configuration from the compressed configuration.

The transition between the compressed configuration and the extended configuration is illustrated in FIG. 16C. Once the lead 103 has fully transitioned to the extended configuration, as shown in FIG. 16D, the electrodes 130, which were oriented radially in the compressed configuration, become oriented in the same direction.

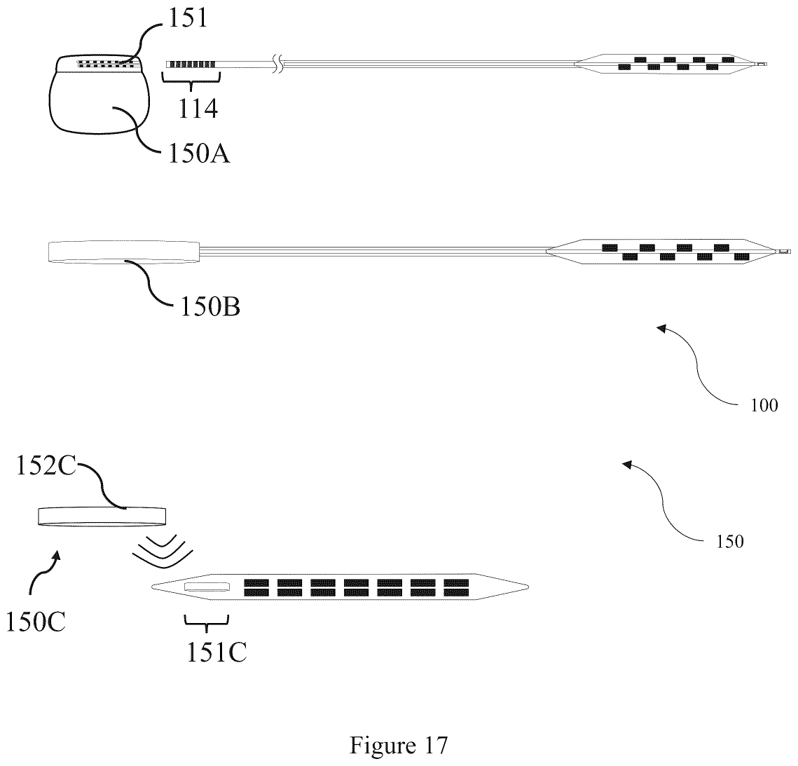

FIG. 17 schematically shows embodiments of the electrophysiology leads system 100 including a controller 150, which may be configured to transmit electrical signals to target tissue, or receive electrical signals from the target tissue. The controller 150 may be configured to stimulate the target tissue, which may include generating an electrical signal and transmitting the electrical signal to the target tissue by way of the lead 103. The controller 150 may generate the electrical signal using a target voltage, target current, target frequency, target pulse-width, each of which may be a fixed value, varying value, or pulsing value. The electrical signal may include alternating current, mono-phasic, bi-phasic pulses, or direct current. In some embodiments, the controller 150 includes a pulse generator.

The controller 150 may be configured to receive electrical signals from the target tissue. The received electrical signals may be used to monitor the target tissue. In some embodiments, the controller 150 records the electrical signals.

As shown in the various embodiments, the controller 150 may be mechanically coupled or couplable to the lead 103, embedded in the lead 103, or remote from the lead 103, among other things. Controller 150A is configured to attach to and detach from an electrical connection 114 on longitudinal body of the lead. Because the controller 150A is located outside the anatomical body after the lead 103 is implanted, the controller 150 may be powered by a battery or other energy storage device, among other things.

Controller 150B is configured to be integrated with the longitudinal body and inserted into the anatomical body when the lead 103 is implanted. Since the controller 150B is located inside the anatomical body, the controller 150B may be configured to use a battery or to receive power or to charge wirelessly. For example, the controller 150B may be radio frequency powered.

Controller 150C includes a stimulation controller 152C positioned external to the anatomical body, and a receiver 151C embedded in the lead 103 and configured to wirelessly communicate with the controller 152C. In some embodiments, the receiver 151C receives a charging signal configured to receive power to the transmitter 152C, the charging signal being distinct from the electrical signal transmitted between the stimulation controller 152C and the target neural tissue. The receiver 151C may fit within the lumen 101 of the lead system or may not.

It is contemplated that the various aspects, features, processes, and operations from the various embodiments may be used in any of the other embodiments unless expressly stated to the contrary. Certain operations illustrated may be implemented by a computer executing a computer program product on a non-transient, computer-readable storage medium, where the computer program product includes instructions causing the computer to execute one or more of the operations, or to issue commands to other devices to execute one or more operations to provide a therapy to the body.

While the present disclosure has been illustrated and described in detail in the drawings and foregoing description, the same is to be considered as illustrative and not restrictive in character, it being understood that only certain exemplary embodiments have been shown and described, and that all changes and modifications that come within the spirit of the present disclosure are desired to be protected. It should be understood that while the use of words such as “preferable,” “preferably,” “preferred” or “more preferred” utilized in the description above indicate that the feature so described may be more desirable, it nonetheless may not be necessary, and embodiments lacking the same may be contemplated as within the scope of the present disclosure, the scope being defined by the claims that follow. In reading the claims, it is intended that when words such as “a,” “an,” “at least one,” or “at least one portion” are used there is no intention to limit the claim to only one item unless specifically stated to the contrary in the claim. The term “of” may connote an association with, or a connection to, another item, as well as a belonging to, or a connection with, the other item as informed by the context in which it is used. The terms “coupled to,” “coupled with” and the like include indirect connection and coupling, and further include but do not require a direct coupling or connection unless expressly indicated to the contrary. When the language “at least a portion” or “a portion” is used, the item can include a portion or the entire item unless specifically stated to the contrary. Unless stated explicitly to the contrary, the terms “or” and “and/or” in a list of two or more list items may connote an individual list item, or a combination of list items. Unless stated explicitly to the contrary, the transitional term “having” is open-ended terminology, bearing the same meaning as the transitional term “comprising.”

Various embodiments of the invention may be implemented at least in part in any conventional computer programming language. For example, some embodiments may be implemented in a procedural programming language (e.g., “C”), or in an object oriented programming language (e.g., “C++”) or in machine language. Other embodiments of the invention may be implemented as a pre-configured, stand-alone hardware element and/or as preprogrammed hardware elements (e.g., application specific integrated circuits, micro-controllers, FPGA, and digital signal processors), or other related components.

In an alternative embodiment, the disclosed apparatus and methods (e.g., see the various flow charts described above) may be implemented as a computer program product for use with a computer system. Such implementation may include a series of computer instructions fixed either on a tangible, non-transitory medium, such as a computer readable medium (e.g., a diskette, CD-ROM, ROM, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as FLASH, EEPROM, semiconductor, magnetic, optical or other memory devices, and may be transmitted using any communications technology, such as optical, infrared, microwave, or other transmission technologies.

The embodiments of the invention described above are intended to be merely exemplary; numerous variations and modifications will be apparent to those skilled in the art. Such variations and modifications are intended to be within the scope of the present invention as defined by any of the appended claims. It shall nevertheless be understood that no limitation of the scope of the present disclosure is hereby created, and that the present disclosure includes and protects such alterations, modifications, and further applications of the exemplary embodiments as would occur to one skilled in the art with the benefit of the present disclosure.

What is claimed is:

1. An electrophysiology lead system, comprising:
in a lead comprising a longitudinal body, a substrate, and an electrode, the electrode being configured to deliver or record an electrical signal, the electrode configured to include a nonuniform thickness or a discontinuity, the lead including an extended configuration and a compressed configuration,
the substrate being normally biased toward the extended configuration,
the electrode being configured to be compressed when the lead is in the compressed configuration,
the electrode being configured so that the substrate urges the electrode toward the extended configuration, and
the electrode including a thickness between 2 microns and 200 microns.

2. The electrophysiology lead system of claim 1, wherein the nonuniform thickness comprises an indentation extending partly through the thickness of the electrode, or wherein the discontinuity comprises a void in the electrode extending completely through the thickness of the electrode.

3. The electrophysiology lead system of claim 1, wherein the normal bias of the substrate is a function of elasticity, shape-memory behavior, thermal change, phase change, polymeric change, or volumetric change.

4. The electrophysiology lead system of claim 1, wherein the substrate includes a guide to direct the lead into the compressed configuration in response to the lead being directed into a lumen.

5. The electrophysiology lead system of claim 1, wherein the lead includes a plurality of electrodes including the first electrode, and wherein each of the plurality of electrodes are oriented in a same direction when the lead is in the extended configuration.

6. The electrophysiology lead system of claim 1, wherein the substrate includes an anchor configured to mechanically couple the lead to a biological tissue.

7. The electrophysiology lead system of claim 1, comprising a radiopaque marker coupled to the substrate.

8. A method for electrically coupling a lead and a neural tissue, comprising:
directing the lead into a compressed configuration including compressing a electrode of the lead, the electrode including a nonuniform thickness or a discontinuity, the electrode being configured to deliver an electrical signal, the electrode including a thickness between about 2 microns and 200 microns; and
transitioning the lead from the compressed configuration to an extended configuration, using a substrate of the lead, including extending the electrode, the substrate being normally biased toward the extended configuration.

9. The method of claim 8, wherein the nonuniform thickness comprises an indentation extending partly through the thickness of the electrode, or wherein the discontinuity comprises a void in the electrode extending completely through the thickness of the electrode.

10. The method of claim 8, wherein the normal bias of the substrate is a function of elasticity, shape-memory behavior, thermal change, phase change, polymeric change, or volumetric change.

11. The method of claim 8, wherein directing the lead into the compressed configuration includes directing, using a guide, the lead into the compressed configuration in response to directing the lead into a lumen.

12. The method of claim 8, wherein the lead includes a plurality of electrodes including the first electrode, and wherein transitioning the lead includes at least a portion of the plurality of electrodes are orienting in the same direction.

13. The method of claim 8, comprising coupling the lead to a biological tissue using an anchor of the substrate.

14. The method of claim 8, wherein the substrate includes a radiopaque marker.

15. An electrophysiology lead, comprising:
a longitudinal body;
a substrate; and
an electrode,
the substrate being biased to move the electrode from a compressed configuration to an extended configuration,
the electrode including a plurality of bending zones configured to be flexed in the compressed configuration and relaxed as the substrate moves toward the extended configuration, and
the electrode including a thickness between about 2 microns and 200 microns.

16. The electrophysiology lead device of claim 15, wherein the plurality of bending zones includes an indentation extending partly through the thickness of the electrode, or voids in the electrode extending completely through the thickness of the electrode.

17. The electrophysiology lead device of claim 15, wherein the normal bias of the substrate is a function of elasticity, shape-memory behavior, thermal change, phase change, polymeric change, or volumetric change.

18. The electrophysiology lead device of claim 15, wherein the substrate includes a guide to direct the electrophysiology lead into the compressed configuration in response to directing the electrophysiology lead into a lumen.

19. The electrophysiology lead device of claim 15, wherein the electrophysiology lead includes a plurality of electrodes including the first electrode, and wherein each of the plurality of electrodes are oriented in a same direction when the electrophysiology lead is in the extended configuration.

20. The electrophysiology lead device of claim 15, wherein the substrate includes an anchor configured to couple the electrophysiology lead to a biological tissue.
